(19) United States
(12) Patent Application Publication
(10) Pub. No.: US 2003/0013109 A1
(43) Pub. Date: Jan. 16, 2003

(54) Hairpin sensors using quenchable fluorescing agents

(76) Inventors: Clinton T. Ballinger, Burnt Hills, NY (US); Michael LoCascio, Albany, NY (US); Daniel P. Landry, Clifton Park, NY (US)

Correspondence Address: KENYON & KENYON, 1500 K STREET, N.W., SUITE 700, WASHINGTON, DC, 20005, US

(21) Appl. No.: 10/176,055

(22) Filed: Jun. 21, 2002

Related U.S. Application Data
(60) Provisional application No. 60/299,460, filed on Jun. 21, 2001.

(51) Int. Cl. C12Q 1/68; C12M 1/34

(52) U.S. Cl. 435/6; 435/287.2

(57) ABSTRACT

The present invention provides for a device and method for detecting genetic material. The device includes at least one hairpin sensor or, preferably two or more hairpin sensors, spatially and/or spectrally multiplexed on a conductive or semi-conductive substrate or particle. The at least one hairpin sensor includes a quenchable fluorescing agent bound to a hairpin loop assembly and the hairpin loop assembly includes a probe complementary to a nucleotide sequence of interest. The method includes providing at least one hairpin sensor, exposing the at least one hairpin sensor to a sample of interest, and detecting fluorescence produced by the quenchable fluorescing agent. The fluorescence indicates the binding of a target nucleotide sequence to the complementary probe of the hairpin loop assembly.

### RELATED U.S. APPLICATIONS

This application claims the benefit and incorporates by reference in its entirety, U.S. Provisional application No. 60/299,460 filed Jun. 21, 2001.

### FIELD OF THE INVENTION

The present invention relates to devices and methods for detecting genetic material through the use of nucleotide probes. More specifically, the present invention relates to methods and materials for quantitatively or qualitatively detecting polynucleotide sequences in sample(s) of biological or non-biological material employing nucleotide probes linked to quenchable fluorescing agents.

### BACKGROUND

Biotechnology research, including biological, biomedical, genetic, fermentation, aquaculture, agriculture, forensic and environmental research, demands the ability to identify nucleic acids and other biological agents both inside and outside cells. Typically, fluorescing dyes are used to assist in the detection of such biological compounds but are often of marginal use. The ideal fluorescing dye for most biotechnology applications should have a high signal to noise ratio (e.g. so that small quantities of nucleic acids can be sensitively detected) and should be resistant to bleaching via exposure to a radiation source (e.g. so that the fluorescence does not diminish with exposure). Many cellular components found in biological systems have an auto-fluorescence (inherent fluorescence) in the visible wavelengths and hence contribute to a relatively high background signal in the visible range. The signal in the infrared wavelength range is very small in most biological applications. Hence, dyes that fluoresce in the visible range must overcome the relatively high background noise level, so they must possess a very high fluorescence efficiency.

A variety of dyes useful for staining and identifying nucleic acids in cell-free and/or intracellular assays have been described. For example, a variety of asymmetrical cyanine dyes (Brooker et al., 1942, J. Am. Chem. Soc. 64:199) and thioflavin dyes (U.S. Pat. Nos. 4,554,546 and 5,057,413) that are useful for staining nucleic acids have been described. The non-chimeric asymmetrical cyanine dye sold under the trade name THLAZOLE ORANGE provides particular advantages in quantitatively analyzing immature blood cells or reticulocytes (U.S. Pat. No. 4,883,867) and in preferentially staining nucleic acids of blood-borne parasites (U.S. Pat. No. 4,937,198). Although Thiazole Orange and other thioflavin cyanine dyes are permeable to membranes of many mammalian cells, they are, however, non-permeable to many eukaryotic cells. A variety of other related cyanine dyes have been described that are permeable to living cells only if the cells' membranes have been disrupted (see, U.S. Pat. Nos. 5,321,130 and 5,410,030).

In addition, a variety of dimeric dyes having cationic moieties useful for staining nucleic acids in electrophoretic gels are described in U.S. Pat. Nos. 5,312,921; 5,401,847; 5,565,554; and 5,783,687. Substituted asymmetric cyanine dyes capable of permeating membranes of a broad spectrum of both living and dead cells have also been described (see, U.S. Pat. No. 5,436,134).

Many of the aforementioned dyes are toxic, have marginal photostability, do not give sufficient sensitivity or require expensive laser systems to induce fluorescence. Many of these dyes, for example, only fluoresce under the influence of a green laser source, which is very expensive. In addition, green laser light often induces auto-fluorescence in some cellular components contributing further to the high background signal and hence decreasing sensitivity. Most dyes emit light with a broad emission spectrum, thus limiting the number of tests that can be performed in a single assay. It is difficult to discriminate the fluorescence associated with a particular dye given the high background and the broad emission spectra of the various dyes in a single test. Many researchers are limited to five or so fluorescing dyes in a single assay, which means the test is limited to a five-fold multiplexing. This is cumbersome since there is often a need to detect dozens of biological agents in a single assay.

Recently, quantum dots have been used as replacements for the traditional molecular fluorescing dyes. They have the advantage of photostability along with a very narrow emission spectrum. The quantum dot applications have been focused on materials that fluoresce in the visible range. In addition, visible spectrum quantum dots have been used for in vitro genetic tagging by using tags suspended in an aqueous solution.

The biochip industry has recently started using miniaturization and integration, similar to computer chip manufacturers, to develop entire assay systems on a single support. These microarrays or “labs on a chip” have been used to revolutionize genomics, drug development, clinical diagnostics and environmental monitoring in much the same way microprocessors revolutionized the computer industry. These microarrays give higher throughput, lower cost, portability and automation than the traditional bio-chemical assay methods. Because these biochips often have used traditional fluorescing dyes, however, they have allowed for only limited spectral multiplexing because of the high background noise and broad emission spectra, for example, of fluorescing dyes. Further, prior microarrays have provided limited spatial multiplexing as the number of molecules that can be identified in a single assay is limited by the number of differentiable locations on the array.

In addition, many microarray approaches involve the primary nucleotide probe being bound to the support and require that the sample be labeled with a fluorescing dye or that a secondary probe be labeled. Such a device is not readily field-deployable as it is not convenient or practical to label a test sample or a secondary probe in a field setting. Therefore, there exists a need for an improved microarray sensor that allows for spectral and/or spatial multiplexing and is also field-deployable.

### SUMMARY OF THE INVENTION

The inventive methods and products disclosed herein will be useful in many medical, industrial, laboratory, and field applications facilitating the detection of many different nucleotide sequences in a single assay. The present invention involves techniques of nucleotide hybridization, labeling with quenchable fluorescing agents, microarray patterning, and spectral and spatial multiplexing.

The present invention provides a hairpin sensor comprising a hairpin loop assembly and a quenchable fluorescing agent. The hairpin loop assembly includes a complementary probe positioned between a first inverse repeat arm and a second inverse repeat arm. The quenchable fluorescing agent is joined directly or indirectly to the end of the second inverse repeat arm opposite the complementary probe.

The present invention also provides for a microarray of hairpin sensors comprising a hairpin sensor or, preferably, two or more hairpin sensors immobilized on a support. The support is capable of quenching the quenchable fluorescing agent. Preferably, the microarray comprises two or more hairpin sensors having complementary probes specific for different target nucleotide sequences and the hairpin sensors are arranged in a spatially-defined pattern to provide for spatial multiplexing. Alternatively, the microarray comprises two or more hairpin sensors having complementary probes specific for different target nucleotide sequences and respective quenchable fluorescing agents that emit different fluorescing wavelengths to provide for spectral multiplexing. More preferably, the microarray further comprises two or more hairpin sensors having two or more complementary probes specific for different target nucleotide sequences and respective quenchable fluorescing agents that emit different fluorescing wavelengths for spectral multiplexing, wherein the hairpin sensors are arranged in a spatially-defined pattern to provide for both spatial and spectral multiplexing.

The present invention also provides a method of detecting a target nucleotide sequence in a sample comprising providing a microarray. The method further provides exposing the microarray to a sample of interest and detecting fluorescence produced by a quenchable fluorescing agent(s). The fluorescence of the agent(s) indicates the binding of a target nucleotide sequence to the respective complementary probe(s). If a microarray comprising two or more hairpin sensors is utilized, in one embodiment, the microarray is arranged in a spatially defined pattern on the support and the target nucleotide sequence is identified by the location of the complementary probe to which the target nucleotide sequence binds.

The present invention also provides a kit for detecting a target nucleotide sequence in a sample comprising an at least one hairpin sensor and a support. The support is capable of quenching the quenchable fluorescing agent of the at least one hairpin sensor.

The present invention additionally provides for a hairpin sensor system comprising a hairpin sensor assembly or, preferably, two or more hairpin sensor assemblies. A hairpin sensor assembly comprises a hairpin loop assembly bound at one end to a quenchable fluorescing agent and bound at another end to a particle. The particle is capable of quenching the quenchable fluorescing agent. The hairpin loop assembly is characterized by a complementary probe positioned between a first inverse repeat arm and a second inverse repeat arm. The particle is attached, directly or indirectly, to the end of the first inverse repeat arm opposite the complementary probe. The quenchable fluorescing agent is joined, either directly or indirectly, to the end of the second inverse repeat arm opposite the complementary probe. When the hairpin sensor system comprises two or more hairpin sensor assemblies, preferably, two or more of the hairpin sensor assemblies have different quenchable fluorescing agents that emit different fluorescence wavelengths.

The present invention also provides a method for detecting a target nucleotide sequence in a sample. The method includes providing a hairpin sensor system. The method further comprises exposing the hairpin sensor system to a sample of interest and detecting fluorescence produced by a quenchable fluorescing agent(s). The fluorescence of the agent(s) indicates the binding of a target nucleotide sequence(s) to the respective complementary probe(s).

### BRIEF DESCRIPTION OF DRAWINGS

FIG. 1 depicts an embodiment of a hairpin sensor according to the present invention.

FIG. 2 depicts an embodiment of a hairpin sensor immobilized on a support according to the present invention.

FIG. 3 depicts a microarray of hairpin sensors immobilized on a support according to the present invention.

FIG. 4 depicts a hairpin sensor including a first and second spacer according to the present invention.

FIG. 5 depicts a hairpin sensor including a double-stranded first spacer according to the present invention.

FIG. 6 depicts a hairpin sensor including a ligand positioned between hairpin loop assembly and quenchable fluorescing agent according to the present invention.

### DETAILED DESCRIPTION OF THE INVENTION

The present invention relates to the detection of genetic material by a hairpin sensor wherein a hairpin sensor includes a quenchable fluorescing agent bound to a hairpin loop assembly. The present invention also relates to a microarray comprising a hairpin sensor or, preferably, two or more hairpin sensors immobilized on a support wherein at least two complementary probes and/or two quenchable fluorescing agents are different. The present invention additionally relates to a hairpin sensor system comprising a hairpin sensor assembly or preferably, two or more hairpin sensor assemblies, wherein a hairpin sensor assembly includes a hairpin loop assembly bound at one end to a quenchable fluorescing agent and bound at another end to a particle capable of quenching the fluorescing agent.

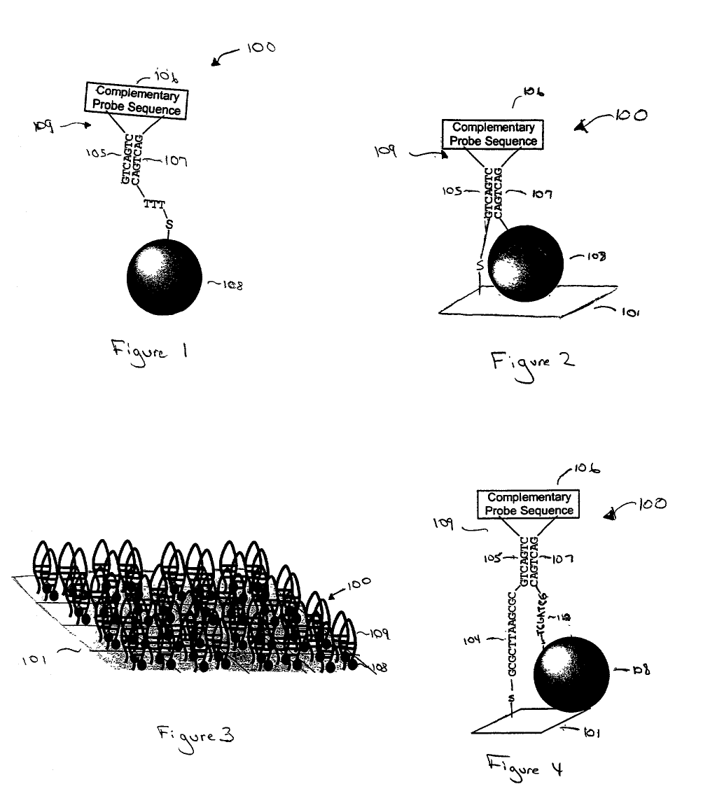

As depicted in FIG. 1, the present invention contemplates a hairpin sensor 100, whether individually or part of a microarray, assembly, or system, generally including a hairpin loop assembly 109 joined to a quenchable fluorescing agent 108. The hairpin sensor is adapted to be bound to a substrate or a particle, both capable of quenching the quenchable fluorescing agent. Hairpin loop assembly 109 may comprise single-stranded RNA or single-stranded or partially double-stranded DNA. Quenchable fluorescing agent 108 preferably comprises a semiconductor nanocrystal, but may also comprise a quenchable fluorescing dye such as, for example, rhodamine-B. Hairpin loop assembly 109 comprises a complementary probe 106 positioned between two nucleotide sequences 105/107, which are capable of forming an inverse repeat sequence when bound together. Quenchable fluorescing agent is bound, either directly or indirectly, to nucleotide sequence 107 and nucleotide sequence 105 is capable of being bound, either directly or indirectly, to a support or particle. Complementary probe 106 is capable of binding specifically and avidly to a specific nucleotide sequence, which is referred to as its “target sequence” herein. In the absence of a hybridized target sequence, the inverse repeat arms 105/107 are non-covalently bound via hydrogen bonds and a hairpin loop forms with complementary probe 106 forming the loop of the hairpin loop. The lengths of the inverse repeat arms 105/107 and complementary probe 106 are preferably selected such that if a target sequence is introduced to hairpin sensor 100, a hybridization bond between inverse repeat arms 105/107 would be less energetically favorable than a hybridization bond between complementary probe 106 and the target sequence. Therefore, once a target sequence binds to complementary probe 106, the bonds between inverse repeat arms 105/107 break and the hairpin loop unfolds. In one embodiment, as seen in FIG. 2, hairpin sensor 100 is immobilized to a support 101 capable of quenching quenchable fluorescing agent 108 when agent 108 is in close contact with support 101. In another embodiment, a conductive or semi-conductive particle is attached to hairpin sensor 100. The conductive particle is capable of quenching agent 108 when agent 108 is in close contact with the particle.

The present invention also contemplates, in general, a microarray comprising a hairpin sensor 100 or, preferably, two or more hairpin sensors 100 immobilized to a support as depicted in FIG. 3. In the absence of a hybridized target sequence, inverse repeat sequences 105/107 are non-covalently bound forming a closed hairpin loop. In the hairpin formation, quenchable fluorescing agent 108 is forced close to support 101 such that fluorescence is prohibited or quenched. Once a target sequence binds to complementary probe 106, the bonds between inverse repeat arms 105/107 break and the hairpin loop unfolds. The unfolding removes quenchable fluorescent agent 108 from its close proximity to support 101 and quenchable fluorescing agent 108 fluoresces. In one embodiment where the microarray comprises two or more hairpin sensors, all of the hairpin sensors have the same complementary probes and the same quenchable fluorescing agents. By “same” complementary probes, what is meant in the context of the present invention, is that the complementary probes have specificity for the same target nucleotide sequence. By “same” quenchable fluorescing agent, what is meant in the context of the present invention, is that the quenchable fluorescing agents emit the same color (the same characteristic fluorescence wavelength emissions). In another embodiment, the microarray includes at least two hairpin sensors having complementary probes that are different yet all the quenchable fluorescing agents are the same. By “different” complementary probes, what is meant in the context of the present invention, is that the complementary probes have specificity for different target nucleotide sequences. In this embodiment, the hairpin sensors of the microarray are arranged in a spatially-defined pattern and the identification of the complementary probe of interest, and hence the target sequence to which it binds, is determined by the location of the bound complementary probe on the support. In another embodiment, the microarray includes at least two hairpin sensors having complementary probes that are different and respective quenchable fluorescing agents that are different. By “different” quenchable fluorescing agent, what is meant in the context of the present invention is quenchable fluorescing agents that emit different colors (different fluorescence wavelength emissions). In this embodiment, the identification of the complementary probe of interest and hence the target sequence to which it binds, is determined by the fluorescence color emitted by the quenchable fluorescing agent of the bound probe.

The present invention also contemplates, in general, a hairpin sensor system comprising a hairpin sensor assembly or, preferably, two or more hairpin sensor assemblies wherein a hairpin sensor assembly comprises a hairpin sensor bound to a conductive or semi-conductive particle. The particle is capable of quenching the hairpin sensor when the hairpin loop assembly is in a closed position. This embodiment is particularly useful when it is desired to use hairpin sensors in a solution or gel. When the hairpin sensor system comprises two or more hairpin sensor assemblies, in one embodiment, the hairpin sensor system includes hairpin sensor assemblies all having the same complementary probes and the same quenchable fluorescing agents. In another embodiment, the hairpin sensor system includes at least two hairpin sensor assemblies having different complementary probes with different respective quenchable fluorescing agents.

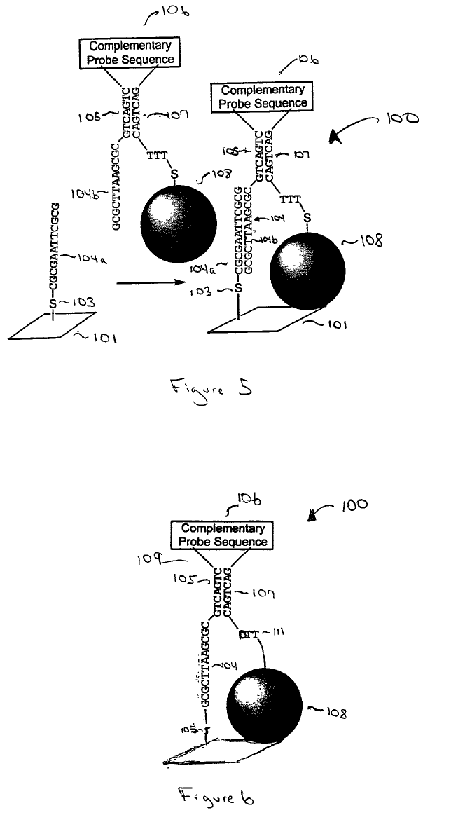

With respect to particular details of the hairpin sensor 100, whether individually or part of a microarray, assembly, or system, hairpin sensor 100 may include a first and/or second spacer as seen in FIG. 4. First spacer 104 is joined to the end of first inverse repeat arm 105 opposite complementary probe 106 and second spacer 110 is joined to second inverse repeat arm 107 opposite complementary probe 106. The spacer(s) function to properly align quenchable fluorescing agent 108 at a distance from support 101 to achieve quenching when hairpin sensor 100 is closed and emission when hairpin sensor 100 is open. The spacer(s) also function to properly position complementary probe 106 such that it can bind to the target sequence. First spacer 104 and/or second spacer 110 may comprise, for example, single-single stranded RNA or single or double-stranded DNA. For example, as seen in FIG. 5, first spacer 104 comprises complementary double-stranded nucleotide strands, 104a and 104b. Strand 104a is immobilized on support 101 or a particle (not shown) via a thiol group 103 and strand 104b is joined to first arm of inverse repeat 105. When strand 110a comes into contact with strand 110b, strand 110a binds strand 110b via hydrogen bonding between the complementary base pairs of strand 104a and strand 104b. In embodiments where first spacer 104 and second spacer 110 are present, it is preferable that the two spacers are not complementary to one another.

Complementary probe 106 of hairpin loop assembly 109 may be designed to bind to an entire target nucleotide sequence or to portions of such sequence. Where binding is to a portion of a target sequence, it is preferable that the portion is unique enough that non-specific binding does not occur. It is generally preferable that complementary probe 106 be constructed such that it forms a single-stranded loop along its entire length or along enough of its length that any double-stranded stretches can easily be disrupted. For example, if complementary probe 106 has double-stranded stretches, these stretches should be shorter than the portion of its target nucleotide sequence that is complementary to complementary probe 106. Preferably, complementary probe 106 contains no stretches of nucleotides that are complementary to one another. In addition, preferably, complementary probe 106 is not complementary to any of first spacer 104, first inverse repeat arm 105, second inverse repeat arm 107, or second spacer 110.

As mentioned above, the length of the inverse repeat and complementary probe 106 will preferably be selected such that a bond between complementary probe 106 and the target sequence will be more energetically favorable than the bond between the inverse repeat arms 105/107 that bind to form the hairpin loop. Therefore, when complementary probe 106 comes into contact with the target sequence, the hairpin loop opens. To ensure that the hairpin loop opens, the relative lengths of complementary probe 106 and the inverse repeat arms 105/107 may be selected so that a certain desired degree of complementarity of binding between complementary probe 106 and its specific target nucleotide is required before the energetic conditions will favor such binding over binding between the inverse repeat arms 105/107.

In certain embodiments, the length of at least one of first spacer 104, first inverse repeat arm 105, complementary probe 106, second inverse repeat arm 107, and second spacer 110, is selected such that the signal produced by quenchable fluorescing agent 108 is quenched when the first and second inverse repeat arms 105/107 non-covalently bind.

In certain embodiments, the length of at least one of first spacer 104, first inverse repeat arm 105, complementary probe 106, second inverse repeat arm 107, and second spacer 110 of hairpin sensor 100 is selected such that the fluorescence probability of the quenchable fluorescing agent 108 is reduced to zero or nearly to zero when first and second arms of inverse repeat arms 105/107 non-covalently bind.

In certain embodiments, the length of first inverse repeat arm 105, complementary probe 106, and second inverse repeat arm 107 are selected such that, when complementary probe 106 and target sequence form a non-covalent bond, the non-covalent bond between first inverse repeat arm 105 and second inverse repeat arm 107 breaks.

In certain embodiments, complementary probe 106 is at least as long or longer than at least one of first inverse repeat arm 105 and second inverse repeat arm 107. With respect to particular lengths of hairpin loop assembly 109, in certain embodiments, at least one of first inverse repeat arm 105 and second inverse repeat arm 107 is between two and 18 nucleotides in length. In other embodiments, at least one of first inverse repeat arm 105 and second inverse repeat arm 107 is between three and 15 nucleotides in length. In still other embodiments, at least one of first inverse repeat arm 105 and second inverse repeat arm 107 is between five and 11 nucleotides in length. In yet other embodiments, at least one of first inverse repeat arm 105 and second inverse repeat arm 107 is seven nucleotides in length.

With respect to particular lengths of optional elements of hairpin sensor 100, in certain embodiments wherein first spacer 104 and second spacer 105 comprise nucleic acids, first spacer 104 is between four and 18 nucleotides in length. In other embodiments, first spacer 104 is between six and 15 nucleotides in length. In yet other embodiments, first spacer 104 is between six and 12 nucleotides in length. In certain embodiments, second spacer 110 is between one and 10 nucleotides in length. In other embodiments, second spacer 110 is between two and 8 nucleotides in length. In still other embodiments, second spacer 110 is 3 nucleotides in length.

Hairpin loop assembly and first and second spacers (if they comprise nucleic acids) may be synthesized using, e.g., automated synthesis machines, which are commercially available. Examples of such machines include the EXPEDITE™ 8909 Nucleic Acid Synthesizer from Applied BioSystems and the ÄKTA OLIGOPILOT DNA/ RNA Synthesizer from Amersham Pharmacia Biotech.

General methods for producing, handling and processing nucleic acids and therefore hairpin loop assembly and possibly first and second spacer 104/110 are known in the art. See, e.g., Sambrook, J., Fritsch, E. F. and Maniatis, T. (1989) Molecular Cloning: A Laboratory Manual, 2nd edition, Cold Spring Harbor Laboratory Press.

Hairpin loop assembly 109 is bound, either directly or indirectly, to quenchable fluorescing agent 108. As mentioned above, quenchable fluorescing agent 108 preferably comprises a semiconductor nanocrystal (also referred to as a “quantum dot”). A semiconductor nanocrystal is a nanometer-sized crystal made of semiconductor material and in known in the art. In general, semiconductor nanocrystals can emit multiple colors of light and changing the size or composition of the nanocrystal, for example, changes the color of the fluorescence. A single light source is sufficient to visualize the multiple colors of semiconductor nanocrystals. As is described in more detail below, it is preferable to use semiconductor nanocrystals in the present invention that have various emission wavelengths in a single array to enable spectral multiplexing in the array.

Generally, semiconductor nanocrystals may be prepared in a manner that results in relative monodispersity; e.g., the diameter of the core of the nanocrystal varying approximately less than 10% between semiconductor nanocrystals in the preparation. The semiconductor nanocrystals used in the present invention may be from about 1 nm to about 100 nm in diameter. Processes for producing semiconductor nanocrystals are known in the art. See, e.g., P. T. Guerreiro et al., “PbS Quantum-Dot Doped Glasses as Saturable Absorbers for Mode Locking of a Cr:Forsterite Laser”, Appl. Phys. Lett. 71 (12), Sep. 22, 1997 at 1595; Nozik et al., “Colloidal Quantum Dots of III-V Semiconductors”, MRS Bulletin, February 1998 at 24; and Hao et al., “Synthesis and Optical Properties of CdSe and CdSe/CdS Nanoparticles”, Chem. Mater. 1999, 11 at 3096.

As understood by those skilled in the arts, nanocrystal synthesis may involve tri-octal phosphine oxide (TOPO) as both the solvent in which nanocrystals are grown and as the ligand that controls growth, although other ligand chemistries, such as fatty acids and phosphine oxides have also been used. Typically, monodisperse nanocrystals are prepared by a single, temporally short nucleation event followed by slower growth on the existing nuclei. This may be achieved by the rapid addition of reagents into a reaction vessel containing a hot, coordinating solvent, such as TOPO, followed by a cooling step. Such a procedure is described, for example, in Murray, C. B., “Colloidal synthesis of nanocrystals and nanocrystal superlattices”, IBM J. Res. & Dev., Vol. 45 No. 1, January 2001.

If the nanocrystals contain nonpolar ligands, it is generally preferable to exchange the nonpolar ligands with polar ligands, thereby increasing the solubility of the nanocrystals in a polar solvent, such as water. For example, if the nanocrystals are in a nonpolar solvent, such as TOPO or another synthesis solvent, the nanocrystals are preferably isolated from the nonpolar solvent by centrifugation and washing in a suitable wash solution, such as hexane/methanol. An exchange ligand solution may be prepared by putting an exchange ligand in a polar solvent. Preferably, a base is added so that the solution has a pH greater than 7, and preferably a pH greater than 8. Preferred exchange ligands are capable of binding to the nanocrystal and increasing the solubility of the nanocrystal in a polar solvent. These ligands generally include a functional group such as a thiol group attached to the semiconductor nanocrystal. Preferably, these exchange ligands are bi-functional and include another functional group for attachment of the semiconductor nanocrystal to the hairpin loop assembly. Examples of such bi-functional ligands include mercapto, hydroxyl, amino, nitrile, carboxyl, and carboxylic acid groups or the like. More specific examples of such ligands include alcohols, mercapto alcohols, thiolated amino acids and organic acids. Even more specific examples of such ligands include 3-mercapto propanol, DTT, and cysteine. The nanocrystals having hydrophobic ligands may be combined with the exchange ligand solution, thereby causing the nanocrystals to precipitate out of solution. Preferably, the nanocrystals are suspended in the solution, such as by using an ultrasonic bath. The temperature of the solution is preferably raised to facilitate the exchange reaction between the hydrophobic ligand and the exchange ligand. To reduce or prevent oxidation of the nanocrystal, the exchange reaction is preferably undertaken in an inert gas flow or overpressure that eliminates oxygen from the reaction chamber.

Semiconductor nanocrystals useful in the practice of the invention include, for example, semiconductor of Group IIA metals/Group VIA metals; Group IIB/Group VIA metals; Group IIIA/Group V metals; Group IVA metals, and Group IVA metals/Group VIA metals and alloys of two or more semiconductors of same.

In certain embodiment, semiconductor nanocrystals are passivated. In such embodiments, semiconductor nanocrystals are used in a core/shell configuration wherein a semiconductor nanocrystal forms a core ranging in diameter, for example, from about 1 nm to about 100 nm, with a shell of another semiconductor nanocrystal material grown over the core semiconductor nanocrystal to a thickness of, for example, 1-10 monolayers in thickness.

The shell grown over the core semiconductor nanocrystal may be an inorganic shell (“cladding” or “coating”) uniformly deposited thereon. Cladding can greatly increase the optical cross-section of the core semiconductor, thus decreasing the optical power required for saturation as well as decreasing the relaxation time. An electrically conducting shell material (like a metal) locally increases the light intensity within the core semiconductor, thus enhancing the absorption cross section. A semiconductor shell material acts as a surface passivating agent and reduces the number of trapped states, which increases the absorption cross section. Methods of and materials useful for passivation are known in the art.

With respect to the absorption and emission wavelengths of semiconductor nanocrystals used in the present invention, semiconductor nanocrystals are preferably capable of absorbing radiation over a broad wavelength band. This wavelength band includes the range from gamma radiation to microwave radiation. In addition, it is preferable that these semiconductor nanocrystals have a capability of emitting radiation within a narrow wavelength band of about 40 nm or less, more preferably about 20 nm or less. This permits the simultaneous use of a plurality of differently colored hairpin sensors 100 with different semiconductor nanocrystals without overlap (or with a small amount of overlap) in wavelengths of emitted light when exposed to the same energy source. Both the absorption and emission properties of semiconductor nanocrystals may serve as advantages over dye molecules which have narrow wavelength bands of absorption (e.g. about 30-50 nm) and broad wavelength bands of emission (e.g. about 100 nm) and broad tails of emission (e.g. another 100 nm) on the red side of the spectrum. Both of these properties of dyes impair the ability to use a plurality of differently colored dyes when exposed to the same energy source.

The frequency or wavelength of the narrow wavelength band of light emitted from the semiconductor nanocrystal may be selected according to the physical properties, such as size, of the semiconductor nanocrystal. In particular, the wavelength band of light emitted by the semiconductor nanocrystal, formed using the above embodiment, may be determined by either (1) the size of the core, or (2) the size of the core and the size of the shell, depending on the composition of the core and shell of the semiconductor nanocrystal. For example, a nanocrystal composed of a 3 nm core of CdSe and a 2 nm thick shell of CdS will emit a narrow wavelength band of light with a peak intensity wavelength of 600 nm. In contrast, a nanocrystal composed of a 3 nm core of CdSe and a 2 nm thick shell of ZnS will emit a narrow wavelength band of light with a peak intensity wavelength of 560 nm.

A plurality of alternatives to changing the size of the semiconductor nanocrystals in order to selectively manipulate the emission wavelength of semiconductor nanocrystals exist. These alternatives include: (1) varying the composition of the nanocrystal, and (2) adding a plurality of shells around the core of the nanocrystal in the form of concentric shells. It should be noted that different wavelengths can also be obtained in multiple shell type semiconductor nanocrystals by respectively using different semiconductor nanocrystals in different shells, i.e., by not using the same semiconductor nanocrystal in each of the plurality of concentric shells.

Selection of the emission wavelength by varying the composition, or alloy, of the semiconductor nanocrystal is known in the art. As an illustration, when a CdS semiconductor nanocrystal, having an emission wavelength of 400 nm, is alloyed with a CdSe semiconductor nanocrystal, having an emission wavelength of 530 nm, the wavelength of the emission from a plurality of identically sized nanocrystals may be tuned continuously from 400 nm to 530 nm depending on the ratio of S to Se present in the nanocrystal. The ability to select from different emission wavelengths while maintaining the same size of the semiconductor nanocrystal may be important in applications which require the semiconductor nanocrystals to be uniform in size, or for example, an application which requires all semiconductor nanocrystals to have very small dimensions when used in application with steric restrictions.

With respect to the attachment of hairpin loop assembly 109 to quenchable fluorescing agent 108, as seen in FIG. 6, ligand 111 may be used to bond quenchable fluorescing agent 108 to hairpin loop assembly 109. Ligand 111 may comprise, for example, functional groups such as amino, carboxyl, thiol or hydroxyl groups. If the nanocrystals contain nonpolar ligands, these ligands may be exchanged with polar ligands as discussed above. The polar ligands are preferably bifunctional, with one functional group, such as a mercapto group, thiolated amino acid or organic acid, being capable of binding to a nanocrystal and another functional group, such as a hydroxyl or carboxyl group, being capable of binding to the hairpin loop assembly. Specific examples of functional groups capable of binding to the nanocrystal include 3-mercapto propanol, DTT, and cysteine. In a preferred embodiment, the hairpin loop is functionalized, such as by amination. The functional group of the hairpin loop assembly and the functional group of the ligand are selected such that the two functional groups will react and form a covalent bond. In one embodiment, a carboxyl group will react with an amino group to form an amide bond. In other embodiments, a hydroxyl group will react with an amino group to form a carbamate linkage. The ligand may be activated. For example, the hydroxyl group such as 3-mercapto propanol or DTT can be activated with CDI. In another embodiment, a functional group on either the hairpin loop assembly or the ligand is refunctionalized (“activated”) to promote bonding. For example, a hydroxyl group can be activated with a carbonyl diimidazole to form an imidazole-carbamate group that will react with an amino group to form a carbamate linkage. The addition of catalysts may also be desirable for some reactions. Cysteine for example may be reacted in the presence of DIC/DMAP.

Other suitable methods of attaching quenchable fluorescing agent 109 to hairpin loop assembly 109 are known in the art and are encompassed by the present invention. For example, hairpin loop assembly 109 may also be bound directly to quenchable fluorescing agent 108. For example, in one embodiment, hairpin loop assembly 109 is thiolated and bound to quenchable fluorescing agent 108 via the thiol group.

After hairpin loop assembly is attached to quenchable fluorescing agent 109, the present invention contemplates hairpin sensor being attached to either a support or a conductive particle depending on the particular use of the hairpin sensor.

In either case, in order for the fluorescence probability of the quenchable fluorescing agent 108 to be reduced to nearly zero when the quenchable fluorescing agent is in close proximity to a particle or support, the particle or support should be able to accept a charge. Therefore, supports and particles are preferably formed of conductors, semiconductors, or combinations thereof. Any substance which has the desired property of accepting a charge from quenchable fluorescing agent in close proximity thereto may be used as or as part of a support or particle. Preferably, the substance is also capable of being microfabricated, e.g., of having microfeatures created thereon or thereof.

The support surface or particle may comprise a metal. Exemplary metals include Au, Ag, Cu, Pt, Al, graphite, and indium tin oxide (ITO). Au is a particularly preferred support or particle component. The support surface or particle may comprise a doped or undoped semiconductor. Exemplary semiconductors include Si, Ge, GaAs, GaN, GaP, AlAs, and AlGaAs. The support or particle surface may comprise a conducting organic polymer. Exemplary conducting organic polymers include poly(phenylene vinylene) (PPV) and Poly(1-Methoxy-4-(2-Ethylhexyloxy-2,5-phenylvinylene) (MEH-PPV). The support or particle surface may also comprise two or more of: a metal, a doped or undoped semiconductor, and a conducting organic polymer.

Further, the support or particle may comprise materials that are neither conductors nor semiconductors, provided that these materials are coated with a conductor or semiconductor or otherwise modified such that they can accept a charge. Thus, the support or particle may comprise any material to which molecules may be attached through either covalent or non-covalent bonds. This includes, but is not limited to, Langmuir-Bodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is contemplated.

Methods for attachment of oligonucleotides such as hairpin loop assembly 109 to such supports or particles are well-known in the art. See, e.g., Chan, S.; Fauchet, P. M.; Li, Y.; Rothberg, L. J.; Miller, B. L. Phys. Stat. Sol. A 2000, 182, 541;: Brockman, J. M.; Frutos, A. G.; Corn, R. M. J. Am. Chem. Soc. 1999, 121, 8044. For example, as mentioned above, functional groups such as amino, carboxyl, thiol or hydroxyl groups may be used to attach hairpin loop assembly 109 to support 101. In a preferred embodiment, a thiol group is attached to the 5′ end of the hairpin loop assembly for attachment to the support. An amino group is attached to the 3′ end of the hairpin loop assembly for attachment to quenchable fluorescing agent. As mentioned above, the aminated hairpin loop assembly may be attached to the quenchable fluorescing agent via a carbamate linkage.

The present invention also provides for a microarray comprising a hairpin sensor or, preferably, two or more hairpin sensors immobilized on a substrate. When the microarray comprises two or more hairpin sensors, in one embodiment, at least two of the hairpin sensors comprise complementary probes that are different and respective quenchable fluorescing agents that are the same. This embodiment may be particularly useful for pathogen detection where simply the positive detection of one or more known pathogens is desired. For example, the support may comprise a cuvette, tube or filter and the at least two hairpin sensors are immobilized on the bottom inner surface of the cuvette, tube, or filter. The bottom inner surface on which the hairpin sensors are immobilized comprises a conductive surface that can quench the fluorescing agent when the hairpin sensor is in a closed position. The complementary probes can be synthesized to be the complementary DNA sequences to the DNA sequence of a pathogen of interest. A solution of an isolated target sequence that potentially encodes the pathogen of interest may be exposed to the microarray. Any fluorescence emission would indicate that the target sequence does encode the pathogen of interest. As this microarray does not require any additional separate labeling steps, (for example a test sample or secondary probe need not be labeled) this embodiment is capable of being field deployable to test, for example, trace amounts of anthrax, smallpox, plague and other possible biological weapons in the field.

In an alternative embodiment, of the above-described microarray, at least two of the hairpin sensors comprise complementary probes that are different and respective quenchable fluorescing agents that are the same. In this embodiment, the hairpin sensors of the microarray are arranged in a spatially defined pattern. For example, as seen in FIG. 6, a first hairpin sensor 100a is arranged in a first array, and a second hairpin sensor 100b is arranged in a second array. The first array is different from the second array.

Several methods are known in the art to immobilize hairpin sensors on a support in a spatially-defined pattern. For example, in one embodiment, hairpin sensors are patterned on support by photolithography using a photoreactive protecting group on a coupling agent. Such a technique is disclosed in McGall et al. U.S. Pat. No. 5,412,087, which is incorporated in its entirety herein. In this embodiment, thiolpropionate having a photochemically removable protecting group is covalently coupled to functional groups on the surface of the support. Light of the appropriate wavelength is then used to illuminate predefined regions of the surface according to a predetermined pattern, resulting in photo deprotection of the thiol group. A mask may be used to ensure that photo deprotection only takes place at the desired sites according to the desired pattern. Hairpin sensors including hairpin loop assemblies containing thiol reactive groups, such as maleimides, are then exposed to the support and react with the deprotected regions. The unbound hairpin sensors are then washed away, and the patterning process may be repeated at another location with another type of hairpin sensor. In the context of the present invention, a given “type” of hairpin sensor comprises those hairpin sensors with complementary probes having specificity for the same target nucleotide sequence; different “types” of hairpin sensors with complementary probes have specificities for different target nucleotide sequences.

A similar method of spatially patterning hairpin sensors involves using a 5′-nitroveratryl protected thymidine group linked to an aminated surface via a linkage to the 3′-hydroxyl end of the thymidine group. (Fodor et al. 1991, Science 251:767-773). Photo deprotection of the thymidine derivative allows a phosphoramidite activated monomer (or oligonucleotide) to react at this site. Other methods use a photoactivatable biotin derivative to spatially localize avidin binding. The avidin, by virtue of its ability to bind more than one biotin group at a time, may in turn be used as a means for spatially localizing a biotin-linked oligonucleotide 109 to the surface (Barrett et al. U.S. Pat. No. 5,252,743 and PCT 91/07807). In principle, spatial patterning of the support using the photo deprotection of a caged binding agent may be used for any ligand-receptor pair where one member of the pair is a small molecule capable of being protected by a photolabile group. Other examples of such ligand-receptor pairing include mannose and concanavalin A, cyclic AMP and anti-cAMP antibodies, and tetrahydrofolate and folate binding proteins (U.S. Pat. No. 5,252,743).

In another embodiment, the regions of a support that come into contact with hairpin sensors at each attachment photoactivation step are spatially restricted. This may be done by placing a support on a block containing channels through which hairpin sensors may be pumped, with each channel giving hairpin sensors access to only a small region of the surface of the support. This prevents accidental binding of hairpin loop assemblies to non-photoactivated regions. Furthermore, the channels may be used to permit the simultaneous attachment of several different hairpin sensors to support. In this embodiment, a mask allows for the patterned illumination and consequent photoactivation of several regions of the surface at the same time. If the area surrounding each photoactivated region is segregated from the neighboring region by a channel, then different hairpin sensors may be delivered to these photoactivated regions by pumping each hairpin sensor through a different channel (Winkler et al., U.S. Pat. No. 5,384,261).

The photoactivated regions in the methods described above may be at least as small as 50 mm². It has been shown that >250,000 binding sites per square centimeter is easily achievable with visible light; the upper limit being determined only by the diffraction limit of light (Fodor et al. 1991, Science 251:767-773). Therefore, photoactivation using electromagnetic radiation of a shorter wavelength may be used to generate correspondingly denser binding arrays. If the support is transparent to the incident radiation it may be possible to simultaneously perform this process on a vertical stack of supports, greatly increasing the efficiency of microarray production.

Alternatively, some form of template-stamping is contemplated by the present invention to spatially pattern hairpin sensors on the support. In this embodiment, a template containing the ordered array of hairpin sensors (and possibly manufactured as described above) may be used to deposit the same ordered array on multiple supports.

In a further embodiment, the microarray of hairpin sensors may be formed on the surface of a support by an “ink-jet” method, whereby the hairpin sensors may be deposited by electromechanical dispensers at defined locations. An ink-jet dispenser capable of forming arrays of hairpin sensors with a density approaching one thousand per square centimeter is described in Hayes et al. U.S. Pat. No. 5,658,802.

In a further embodiment, hairpin sensors may be patterned on a support using soft lithography techniques. See, e.g., U.S. Pat. Nos. 6,048,623; 5,900,160.

In addition, many methods are known in the art for creating micropatterns. As an example, automated arrayers are commercially available. Examples of such arrayers include the VIRTAK CHIPWRITER™ Pro by Virtek Biotechnology and the OMNIGRID™ and OMNIGRID™ ACCENT from GeneMachines®. Guidance is also available in the form of publications in patents and in the literature. See, e.g., Hedge, P., et al., Biotechniques (2000) 29(3): 548-562.

In another embodiment of the microarray, at least two of the hairpin sensors comprise complementary probes that are different and respective quenchable fluorescing agents that are different. This embodiment allows for color multiplexing of the hairpin sensors as the detection of target nucleotide sequences is based not on the spatial location of hairpin sensors on the support but on the distinct fluorescence of the quenchable fluorescing agents. When the quenchable fluorescing agents comprise semiconductor nanocrystals, the spectrally multiplexed microarrays of the present invention make use of the wide variety of optically distinguishable fluorescence emissions (e.g., colors) available in semiconductor nanocrystals to perform multiple assays. When the quenchable fluorescing agents comprise a quenchable fluorescing dye, the fluorescing dyes should exhibit distinctly different colors. The at least two hairpin sensors preferably have a specific associated color and are distinguished from each other by color, rather than by distinct and defined location. The at least two hairpin sensors do not have to be in a spatially multiplexed form, as data regarding location is not needed for identification of the target nucleotide sequences that have bound to their complementary probe. The multiplexing ability comes from the characteristic wavelength of the quenchable fluorescing agent that is attached to each hairpin loop assembly to form each hairpin sensor.

Because the distinct location of the at least two hairpin sensors need not be mapped, hairpin sensors can be immobilized in a smaller surface area thereby requiring a smaller sample volume. The ability to use a smaller sample is valuable in many situations, such as those in which only a small quantity of sample is available, or situations in which the sample is toxic or infectious or it is otherwise undesirable to obtain, maintain, or produce (e.g., via culturing) a larger sample. Therefore this embodiment is also useful for detecting pathogens in a sample.

In yet another embodiment of the microarray, both spatial patterning and color multiplexing can be applied. For example, in this embodiment at least two of the hairpin sensors comprise complementary probes that are different with respective quenchable fluorescing agents that are different. In addition, the at least two hairpin sensors of the microarray are arranged in a spatially-defined pattern. This embodiment is particularly useful for applications that favor high-throughput screening, such as drug discovery as there are two levels of multiplexing.

The present invention also contemplates a method for detecting a target nucleotide sequence in a sample. The method includes providing a microarray. The method further provides exposing the microarray to a sample of interest and then detecting fluorescence produced by the quenchable fluorescing agent(s), wherein the fluorescence indicates the binding of a target nucleotide sequence to a complementary probe(s). For the embodiments where hairpin sensors of the microarray are arranged in a spatially-defined pattern on the support, the method includes the additional step of identifying the target nucleotide sequence by the location of the complementary probe(s) to which the target nucleotide sequence binds.

The present invention also contemplates a kit for detecting a target nucleotide sequence in a sample. The kit includes a hairpin sensor and a support. The support of the kit is capable of accepting a charge. The kit may include any quenchable fluorescing agent whose fluorescence reduced to zero or nearly zero when the agent is in close contact with the support.

In addition to the microarray, the present invention also contemplates a hairpin sensor system comprising a hairpin sensor assembly or, preferably, two or more hairpin sensor assemblies. The hairpin sensor assembly comprises a hairpin loop assembly bound at one end to a conductive or semi-conductive particle and bound at another end to a quenchable fluorescing agent. In particular, the first inverse repeat arm of the hairpin loop assembly is bound, directly or indirectly, to a particle and the second inverse repeat arm is bound, directly or indirectly, to a quenchable fluorescing agent. The attached conductive or semi-conductive particle acts as a quencher for the quenchable fluorescing agent of the hairpin sensor assembly to which it is coupled. As with hairpin sensors immobilized to a conductive or semi-conductive support, the particle is separated from the semiconductor nanocrystal upon binding of a complementary nucleotide to the complementary probe. Thus, when the quenchable fluorescing agent associated with a particular complementary probe fluoresces, the presence of a nucleotide sequence complementary to the probe sequence is identified.

In one embodiment, the hairpin sensor system includes at least two hairpin sensor systems comprising at least two complementary probes that are the same and respective at least two quenchable fluorescing agents that are the same. As mentioned above in reference to the microarray, this embodiment may be particularly useful for pathogen detection where the simple positive detection of a known pathogen is desired. For example, the hairpin sensor system may be placed in a cuvette, tube or filter. The at least two complementary probes can be synthesized to be the complementary DNA sequences to the DNA sequence of a pathogen of interest. For example, where a pathogen, such as anthrax, is sought to be detected, a probe having a binding sequence that is complementary to a target nucleotide sequence within the anthrax genome may be constructed. A solution of an isolated target sequence that potentially encodes the pathogen of interest may be exposed to the system. Any fluorescence emission would indicate that the target sequence does encode the pathogen of interest. This embodiment is also capable of being field deployable.

In another embodiment, the hairpin sensor system includes at least two hairpin sensor assemblies comprising at least two complementary probes that are different and at least two quenchable fluorescing agents are different. This embodiment allows for color multiplexing of the hairpin sensor assemblies. As mentioned in relation to the microarray, the at least two hairpin sensor assemblies preferably emit specific colors and a target nucleotide sequence attaches to a hairpin sensor assembly such that the detection step maps the characteristic quenchable fluorescing agent's fluorescence wavelength to a particular complementary probe.

This system is particularly useful when a solution or a gel are utilized in an assay. For example, the system may be suspended in a gel or allowed to float free in a liquid. In such embodiments, probes according to the present invention are coupled to a particle of conductive or semiconductive material, such as a gold particle. The solution or gel may be in a container suitable for performing assays, such as a tube, cuvette, or well or a plate. In such sensors, the probes are not fixed to a support. Again, the precise locations of the probes need not be known, and there is no need for precise patterning or maintenance of orientation information.

The present invention also contemplates a method for detecting a target nucleotide sequence in a sample. The method includes providing a hairpin sensor system, exposing the system to a sample of interest, and detecting fluorescence produced by the quenchable fluorescing agent(s).

With respect to detection of fluorescence emission once a target nucleotide sequence binds to complementary probe (whether of an individual hairpin sensor, a microarray comprising two or more hairpin sensors, a hairpin sensor assembly, or a system comprising two or more hairpin sensor assemblies), the fluorescence emissions produced by the quenchable fluorescing agent may be read by any suitable device that can measure the wavelength and intensity of the light emitted from the quenchable fluorescing agent. Exemplary such devices are those that collects light from fluorescence emissions, splits it into its wavelength components, and projects the resultant spectrum onto a detector.

If the quenchable fluorescing agent comprises a semiconductor nanocrystal, the nature of such a nanocrystal is such that a single excitation source can be used to excite all fluorescing colors. Further, excitation need not be with a single wavelength, but may be with a range of wavelengths; for example, a lamp may be used. Preferably, the light source used to excite the semiconductor nanocrystals in a given assay, device or composition emits light of a shorter wavelength (higher energy) than the fluorescent emission wavelength of the nanocrystal having the shortest emission wavelength of all nanocrystals in that given assay, device or composition. For example, an ultraviolet light source, such as a UV light emitting diode (LED), mercury discharge lamp, etc., can be used as the source of excitation for semiconductor nanocrystals that fluoresce in the visible portion of the spectrum. For nanocrystals that fluoresce in the infrared (longer wavelength, lower energy) portion of the spectrum, any form of visible or ultraviolet light source can be used as the source of excitation.

It is particularly preferable that the longest wavelength emitted from the excitation source used in a given system have a shorter wavelength than the shortest wavelength emitted by a nanocrystal in that system. Such embodiments help to prevent confusion between the light emitted by the excitation source and the light emitted by the nanocrystals, thereby increasing the system's sensitivity.

Detection of fluorescence emissions from hairpin sensors according to the present invention comprising hairpin sensors localized (e.g., immobilized, in solution, or in a gel) in one area (e.g., in a single cuvette, well, tube or plate) may be accomplished using a system that can measure the fluorescence spectrum from a single area (single spot) of a spectrally multiplexed assay. The excitation light can be delivered via an overhead lamp or from underneath (assuming the support is transparent or the assay is in a solution that is at least partially transparent to the excitation light), or delivered to the assay via an optical fiber.

The light emitted from the assay (due to fluorescence emissions from semiconductor nanocrystals) is directed though an optical pupil and onto an optical component, such as a prism or diffraction grating, that separates light into its constituent spectral wavelength components. The light emitted from the assay can be directed to the prism/grating in any suitable manner, such as via an optical fiber or through simple free space propagation. The prism/gratings separate the light into its constituent spectral wavelength components whereby each wavelength component is transmitted or reflected at a slightly different angle.

The emitted light that has been separated into its constituent wavelengths is shone onto a linear detector array, wherein each detector within the linear detector array is illuminated by (and hence detects) one wavelength component of the spectrum. Each detector in the linear detector array measures the intensity of the light falling on it, and because the light falling on an individual detector is of a particular wavelength the array can determine the fluorescence spectra of the single area assay. Examples of suitable detector arrays include, for example, photomultiplier tube arrays, charged couple device arrays, CMOS photodetector arrays, and microbolometer arrays (for infrared detection). In a similar manner a linear arrangement of spots can be detected by collecting the fluorescing light, splitting into its wavelength component and projected it onto a two-dimensional detector array.

When the hairpin sensors are localized in a one area, the light emitted from the assay is directed through an optical pupil and onto an optical component, such as a prism or diffraction grating, that separates light into its constituent spectral wavelength components. The light emitted from the assay can be directed to the prism/grating in any suitable manner, such as via an optical fiber or through simple free space propagation. The prism/gratings separate the light into its constituent spectral wavelength components whereby each wavelength component is transmitted or reflected at a slightly different angle.

The emitted light that has been separated into its constituent wavelengths is shone onto a two-dimensional detector array, where each column of detector within the array measures the fluorescence spectrum of a single area (or spot). Each detector in the array measures the intensity of the light falling on it, and because the light falling on an individual detector is of a particular wavelength the array can determine the fluorescence spectra of each of the spectrally multiplexed assays within the linear array of assays.

An exemplary, commercially-available system useful for detection of fluorescence in assays according to the present invention is the OCEAN OPTICS USB2000 portable spectrophotometer. This system is field-portable, simple to use, and can simultaneously detect a plurality of emission wavelengths. Furthermore, small samples, such as those comprising nanoliters of solution, can be easily detected with hand-held detection systems such as the OCEAN OPTICS system.

Due to the controllable, narrow fluorescence spectrum of semiconductor nanocrystals and the wide variety of semiconductor nanocrystals that can be made, complementary probes comprising different binding sequences can be labeled with a number of different semiconductor nanocrystals and can be readily distinguished from one another.

Each color or wavelength of light emitted by the sensor corresponds to a specific semiconductor nanocrystal, and indicates that that nanocrystal is not quenched. The lack of quenching of a given semiconductor nanocrystal indicates that the hairpin loop connected thereto has been opened, thus indicating that a nucleotide sequence that is complementary to the complementary probe has bound to the complementary probe. Thus, the wavelengths of the emitted light indicates which nucleotide sequences are present in the sample to which the sensor has been exposed.

The linewidths of semiconductor nanocrystals are narrow enough to have many different colors (wavelengths) resolved with simple spectrophotometer equipment. Hence, many different pathogens can be detected in a single assay with a volume in the nanoliter range without difficulty. In one embodiment, semiconductor nanocrystals having at least two different colors (wavelengths) are present in a single sensor device according to the present invention. In another embodiment, semiconductor nanocrystals having at least four different colors (wavelengths) are present in a single sensor device according to the present invention. In one embodiment, semiconductor nanocrystals having at least six different colors (wavelengths) are present in a single sensor device according to the present invention. In another embodiment, semiconductor nanocrystals having at least eight different colors (wavelengths) are present in a single sensor device according to the present invention. In yet another embodiment, semiconductor nanocrystals having at least ten different colors (wavelengths) are present in a single sensor device according to the present invention. As discussed further herein, part of the wavelength range may be reserved for false positive indication on particular nucleotide sequences.

The sensors and methods of the present invention are useful for detecting the presence of target sequences in samples. Such samples include biological samples, such as whole blood, serum, urine, saliva, and tissue samples. Target sequences may also be detected in non-biological samples, such as soil and water samples.

It is preferable that target sequences to be detected using the sensors and methods of the present invention be in single-stranded form. Thus, where the target sequence is present as a double-stranded molecule in its natural state, it will be preferable to separate the strands, using methods known in the art, such as heat denaturing, to produce a single-stranded molecule.

Where the target sequence sought to be detected is present in cells or tissues, it will be preferable to release the target sequence from the cells, using art-known methods. In certain embodiments, it may be preferable to isolate the target sequence from a sample before exposing them to arrays of the present invention.

Additionally, where a target sequence sought to be detected is present in its natural states as a large nucleotide, such as genomic DNA, it may be desirable to fragment target sequence before exposing it to the sensors of the present invention. Such fragmentation may be accomplished by art-known methods, such as exposing the target sequence to non-specific or specific endonucleases, such as restriction enzymes.

The detection system used in the sensors and methods of the present invention are extremely sensitive and can therefore detect even very small quantities of the target sequence. Thus, amplification of the target sequence will normally not be necessary. However, should such amplification be desired, it can be accomplished using art known methods, such as PCR. Primers needed to amplify target sequences are readily designed using art-known technology.

Sensors and methods according to the present invention are particularly useful for detecting the presence of pathogens, such as viral, bacterial, parasitic, and fungal pathogens (e.g., anthrax, smallpox, ebola, malaria and the like) in samples of both biological and non-biological origin (e.g., whole blood, serum, urine, saliva, tissue, soil, and water samples).

Particularly useful target sequences include those that are stable with the genome of the pathogen to be detected; in other words, target sequences of the pathogen sought to be detected that are present in a high percentage of individuals, are particularly useful. Conversely, target sequences that are subject to frequent mutations are less useful as target sequences. Sequences which are stable, as well as those subject to mutation, are know in the art and can also be determined experimentally.

Particularly useful target sequences also include those that are unique to the pathogen sought to be detected, or that are at least uncommon or distinct. Uniqueness need not be absolute, but may rather be relative to the pathogens sought to be screened for in a single assay. For example, if it is desired to create a sensor to simultaneously screen for anthrax, smallpox, and polio, a target sequence for each pathogen may be chosen that is not present in the other pathogens to be screened in that given assay.

As it may be useful to fragment pathogen DNA before screening it, it may be useful to choose a target sequence that may be cleaved from the pathogen genomic DNA using one or more restriction endonuclease enzymes. Numerous restriction enzymes, as well as their specificities, are known to the art.

False-positive readings, which often plague detection systems, may also be reduced or eliminated using sensors and methods according to the present invention. Such reduction can be accomplished by creating complementary probes comprising binding sequences that are complementary to nucleotides that are known or thought to trigger false positive readings (such sequences may be referred to herein as “negative indicator sequences”). If fluorescence from a semiconductor nanocrystal associated with a complementary probe that binds to a negative indicator sequence is observed, this will be an indication that any positive result observed may be a false positive.

A complementary probe having a binding sequence complementary to a negative indicator sequence may be designed for each pathogen sought to be detected using a given sensor. Thus, a sensor having complementary probes specific for anthrax, smallpox, and plague may also comprise probes specific for one or negative indicator sequences associated with one or more of anthrax, smallpox, and plague.

The examples that follow are set forth to aid in understanding the invention but are not intended to, and should not be construed to, limit its scope in any way. The examples do not include detailed descriptions of conventional methods. Such methods are well known to those of ordinary skill in the art and are described in numerous publications.

### EXAMPLES

### Example 1

1.1 Preparation of the Gold Support

A single-stranded DNA oligonucleotide consisting of the sequence 5′-CGCGAATTCGCG-3′ is synthesized in such a manner as to provide a 5′-thiol derivative. This is spotted on a gold support using a commercial microarrayer using methods standard in the art.

1.2 Synthesis and Annealing of the Probe Oligonucleotide

A single-stranded DNA oligonucleotide consisting of the sequence 5′-TTTCAGTCAG-“complementary probe”-CTGACTGCGCGAATTCGCG-3′ is synthesized in such a manner as to provide a 5′-thiol derivative, where “complementary probe” indicates a DNA sequence chosen to complement the sequence one desires to detect.

For example, construction of a sensor for the detection of HIV could employ one of a number of sequences from the HIV genome.

For the detection of: 5′-agatggaaaccaaaaatgat-3′ (Derived from HIV protease; Cinque, P. et al., AIDS Res. Hum. Retroviruses 17 (5), 377-383 (2001)), [complementary probe] would consist of 5′-atcatttttggtttccatct-3′.

For the detection of: 5′-gcaccagggaaagggtcaga-3′ (Derived from reverse transcriptase; Cilla, G. et al., AIDS Res. Hum. Retroviruses 17 (5), 417-422 (2001)), [complementary probe] would consist of 5′-tctgaccctttccctggtgc-3′.

This DNA oligonucleotide is first conjugated to a water-soluble, passivated nanocrystal (Au, CdSe, CdS, or PbS) using methods which are standard in the art. A solution of this nanocrystal-conjugated DNA oligonucleotide in tris buffer, pH 7.4, 100 mM KCl, is next deposited on the prepared gold support at a temperature of 60 degrees C. The temperature is lowered from 60 degrees C. to ambient (approximately 23 degrees C.) over a one-hour period to permit annealing of the nanocrystal-conjugated probe oligonucleotide to the oligonucleotide-functionalized gold support.

### Example 2

Thiolated, aminated oligonucleotide hairpins (5′C6thiol-GCGAGTTTTTTTTTTTTTTTCTCGC-3′ AminoC7) are labeled with rhodamine B via their amine moieties to form hairpin sensors. The hairpin sensors are then immobilized to a gold surface via their thiol moieties.

When excited at the appropriate wavelength in water, the immobilized hairpin sensors show no fluorescence at baseline (FIGS. A&B). When excited at the appropriate wavelength in a concentrated solution of complementary DNA (5′AAA AAAAAAAAAAAA3′), the fixed labeled hairpins fluoresce brightly (FIG. D) and can be seen with confocal fluorescence microscope (FIG. C). This experiment is done at room temperature using an inexpensive, readily available buffer (phosphate buffered saline solution, pH 7.4). The results demonstrate that immobilization of hairpin sensors to a gold surface suppresses their fluorescence at baseline. They also confirm that this suppression is reversed by hybridization to a complementary template. They show that this reversal of suppression takes place instantaneously in readily achievable conditions.

### Example 3

Thiolated, aminated oligonucleotide hairpins (5′C6thiol-GCGAGTTTTTTTTTTTTTTTTTTTTCTCGC-3′AminoC7) are labeled with rhodamine B via their amine moieties to form hairpin sensors. The hairpin sensors are then immobilized to a gold surface via their thiol moieties.

The hairpin sensors are a) hybridized to a 10-fold dilution series of 5′AAAAAAAAAAAAAAAAAAAA3′ templates (107 to 1 molecules per 10 μl of template in water) or b) hybridized to a 10-fold dilution series of 5′AAAAAAAAAAAAAAAAAAAA3′ templates (107 to 1 molecule of template, plus 1 μg/ml fish sperm DNA, in water).

These experiments facilitate determination of the sensitivity of the method for detecting known (and vanishingly small) quantities of template in water and in the presence of irrelevant DNA.

### Example 4

Thiolated, aminated oligonucleotide hairpins (5′GCGAGTTTTTTTTTTTTTTTTTTTTCTCGC3′ AminoC7) are labeled with CdSe semiconductor nanocrystals via their amine moieties to form hairpin sensors. While in liquid phase, the hairpin sensors are hybridized to unlabelled template DNA (5′AAAAAAAAAAAAAAAAAAAA3′ or fish sperm DNA control) that has been fixed to glass surfaces. Following hybridization, the glass surfaces are washed. The surface are then be exposed to ultraviolet light.

This experiment confirms that the hairpin sensors hybridize specifically, can be washed off when not hybridized, and retain their fluorescent properties.

### Example 5

Thiolated, aminated oligonucleotide hairpins (5′6thiolGCGAGTTTTTTTTTTTTTTTTTTTTCTCGC3′AminoC7) are labeled with CdSe semiconductor nanocrystals via their amine moieties to form hairpin sensors. The hairpin sensors are then immobilized to a gold surface via their thiol moieties.

The immobilized hairpin sensors are then be hybridized to unlabeled template DNA (5′AAAAAAAAAAAAAAAAAAAA3′ or fish sperm DNA control) in liquid phase. Following hybridization, the surface is exposed to ultraviolet light. Fluorescence is evaluated using a fluorometer. This experiment confirms the functionality of the fluorometer for reading assays according to the present invention.

We claim:

1. A hairpin sensor comprising:
a hairpin loop assembly including,
a complementary probe positioned between a first inverse repeat arm and a second inverse repeat arm; and
a quenchable fluorescing agent joined, directly or indirectly, to the end of the second inverse repeat arm of the hairpin loop assembly opposite the complementary probe.

2. The hairpin sensor of claim 1, further comprising a functional group joined to the end of the first inverse repeat arm opposite the complementary probe, the functional group selected from the group consisting of amino, carboxyl, thiol, and hydroxyl.

3. The hairpin sensor of claim 1, further comprising a first spacer joined to the end of the first inverse repeat arm opposite the complementary probe.

4. The hairpin sensor of claim 3, further comprising a functional group joined to the end of the first spacer opposite the first inverse repeat arm, the functional group selected from the group consisting of amino, carboxyl, thiol, and hydroxyl.

5. The hairpin sensor of claim 1, further comprising a ligand positioned between the second inverse repeat arm and the quenchable fluorescing agent, the ligand selected from the group consisting of mercapto, hydroxyl, amino, nitrile, and carboxyl, carboxylic acid, organic acid, and amino acid.

6. The hairpin sensor of claim 1, further comprising a second spacer positioned between the second inverse repeat arm and the quenchable fluorescing agent.

7. The hairpin sensor of claim 6, further comprising a ligand positioned between the second spacer and the quenchable fluorescing agent, the ligand selected from the group consisting of mercapto, hydroxyl, amino, nitrile, and carboxyl, carboxylic acid, organic acid, and amino acid.

8. The hairpin sensor of claim 1, wherein the quenchable fluorescing agent comprises a semiconductor nanocrystal.

9. The hairpin sensor of claim 1, wherein the quenchable fluorescing agent comprises a rhodamine B-labeled dye.

10. A microarray comprising:
at least one hairpin sensor including,
a hairpin loop assembly characterized by,
a complementary probe positioned between a first inverse repeat arm and a second inverse repeat arm, the end of the first inverse repeat arm opposite the complementary probe bound, directly or indirectly, to a support; and
a quenchable fluorescing agent joined directly or indirectly to the end of the second inverse repeat arm of the hairpin loop assembly opposite the complementary probe.

11. The microarray of claim 10, wherein the support is capable of accepting a charge.

12. The microarray of claim 10, wherein the at least one hairpin sensor comprises two or more hairpin sensors.

13. The micro array of claim 12, wherein the two or more hairpin sensors include complementary probes that are the same and respective quenchable fluorescing agents that are the same.

14. The microarray of claim 12, wherein the two or more hairpin sensors include complementary probes that are different and respective quenchable fluorescing agents that are the same.

15. The microarray of claim 14, wherein the two or more hairpin sensors are arranged in a spatially-defined pattern.

16. The microarray of claim 10, wherein the two or more hairpin sensors include complementary probes that are different and respective quenchable fluorescing agents that are different.

17. The microarray of claim 16, wherein the two or more hairpin sensors are arranged in a spatially-defined pattern.

18. A method for detecting a target nucleotide sequence in a sample comprising:
providing at least one hairpin sensor immobilized on a substrate, the at least one hairpin sensor comprising
a hairpin loop assembly including,
a complementary probe positioned between a first inverse repeat arm and a second inverse repeat arm, the end of the first inverse repeat arm opposite the complementary probe bound, directly or indirectly, to a support; and
a quenchable fluorescing agent joined, directly or indirectly, to the second inverse repeat arm of the hairpin loop assembly;
exposing the at least one sensor to a sample of interest; and
detecting fluorescence produced by the quenchable fluorescing agent,
wherein the fluorescence indicates the binding of the target nucleotide sequence to the complementary probe.

19. The method of claim 18, wherein the at least one hairpin sensor comprises two or more hairpin sensors.

20. The method of claim 19, wherein the two or more hairpin sensors are arranged in a spatially-defined pattern on the support.

21. The method of claim 20, further comprising identifying the target nucleotide sequence by the location of the complementary probe to which the target nucleotide sequence binds.

22. The method of claim 19, wherein the two or more hairpin sensors include complementary probes that are different.

23. The method of claim 19, wherein the two or more hairpin sensors include quenchable fluorescing agents that are different.

24. A kit for detecting a target nucleotide sequence in a sample comprising:
a hairpin sensor characterized by,
a hairpin loop assembly including,
a complementary probe positioned between a first inverse repeat arm and a second inverse repeat arm; and
a quenchable fluorescing agent joined, directly or indirectly, to the second inverse repeat arm of the hairpin loop assembly; and
a support.

25. The kit of claim 24, wherein the support is capable of accepting a charge.

26. A hairpin sensor system including at least one hairpin sensor assembly, the at least one hairpin sensor assembly comprising:
a hairpin loop assembly including
a complementary probe positioned between a first inverse repeat arm and a second inverse repeat arm, wherein the end of the first inverse repeat arm opposite the complementary probe is bound, directly or indirectly, to a particle; and
a quenchable fluorescing agent joined, directly or indirectly, to the end of the second inverse repeat arm opposite the complementary probe.

27. The hairpin sensor system of claim 26, wherein the particle is conductive or semi-conductive.

28. The hairpin sensor system of claim 26, further comprising a functional group joined to the end of the first inverse repeat arm opposite the complementary probe, the functional group selected from the group consisting of amino, carboxyl, thiol, and hydroxyl.

29. The hairpin sensor system of claim 26, further comprising a first spacer joined to the end of the first inverse repeat arm opposite the complementary probe.

30. The hairpin sensor system of claim 29, further comprising a functional group joined to the end of the first spacer opposite the first inverse repeat arm, the functional group selected from the group consisting of amino, carboxyl, thiol, and hydroxyl.

31. The hairpin sensor system of claim 26, further comprising a ligand positioned between the second inverse repeat arm and the quenchable fluorescing agent, the ligand selected from the group consisting of mercapto, hydroxyl, amino, nitrile, and carboxyl, carboxylic acid, organic acid, and amino acid.

32. The hairpin sensor system of claim 26, further comprising a second spacer positioned between the second inverse repeat arm and the quenchable fluorescing agent.

33. The hairpin sensor system of claim 32, further comprising a ligand positioned between the second spacer and the quenchable fluorescing agent, the ligand selected from the group consisting of mercapto, hydroxyl, amino, nitrile, and carboxyl, carboxylic acid, organic acid, and amino acid.

34. The hairpin sensor system of claim 26, wherein the quenchable fluorescing agent comprises a semiconductor nanocrystal.

35. The hairpin sensor system of claim 26, wherein the quenchable fluorescing agent comprises a rhodamine B-labeled dye.

36. The hairpin sensor system of claim 26, wherein the at least one hairpin sensor assembly comprises two or more hairpin sensor assemblies.

37. The hairpin sensor system of claim 36, wherein the two or more hairpin sensor assemblies include complementary probes that are the same and respective quenchable fluorescing agents that are the same.

38. The hairpin sensor system of claim 36, wherein the two or more hairpin sensors include complementary probes that are different and respective quenchable fluorescing agents that are different.

39. A method for detecting a target nucleotide sequence in a sample comprising:
providing a hairpin sensor system, the hairpin sensor system including at least one hairpin sensor assembly, the at least one hairpin sensor assembly comprising:
a hairpin loop assembly including,
a complementary probe positioned between a first inverse repeat arm and a second inverse repeat arm, wherein the first inverse repeat arm is bound, directly or indirectly, to a particle; and
a quenchable fluorescing agent joined, directly or indirectly, to the
second inverse repeat arm of the hairpin loop assembly;
exposing the hairpin sensor system to a sample of interest; and
detecting fluorescence produced by the quenchable fluorescing agent attached to the bound complementary probe.

40. The method of claim 39, wherein the at least one hairpin sensor assembly comprises two or more hairpin sensor assemblies.
